(19) United States
(12) Patent Application Publication
(10) Pub. No.: US 2005/0107795 A1
(43) Pub. Date: May 19, 2005

(54) Bone plating system and method of use

(76) Inventors: John Morris, Beechwood, NJ (US); Lawrence A. Shimp, Morganville, NJ (US); David Kaes, Toms River, NJ (US); Nelson L. Scarborough, Andover, MA (US)

Correspondence Address: DILWORTH & BARRESE, LLP, 333 EARLE OVINGTON BLVD., UNIONDALE, NY, 11553, US

(21) Appl. No.: 10/486,552

(22) PCT Filed: Aug. 9, 2002
(86) PCT No.: PCT/US02/25360

Related U.S. Application Data
(60) Provisional application No. 60/311,531, filed on Aug. 10, 2001.

(51) Int. Cl. A61F 2/30; A61B 17/56; A61B 17/58

(52) U.S. Cl. 606/69

(57) ABSTRACT

A bone plating system includes an external fixation plate fabricated from bone and/or other remodelable materials(s).

### BACKGROUND OF THE INVENTION

1. Field of the Invention

The present invention relates to a bone plating system and, more particularly, to a bone plating system made from cortical bone and used to retain a plurality of bones or bone fragments in fixed relation to each other.

2. Description of Related Art

In repairing bone fractures or stabilizing adjacent vertebrae, it is known to affix a bone plate to the bone pieces utilizing screws to provide for vertebral fusion and/or healing of the fractured bone to occur. Typically, a bone plate is fastened to an exposed surface of the bone or a reconstructed area thereof to immobilize the fracture or vertebral segment thereof, in order for the fractured or reconstructed bone to undergo a healing or fusing process. A typical bone plate is made from a strip of metal such as surgical grade stainless steel, titanium, or other materials having high tensile strength and is provided along its length with a series of screw holes thereby permitting the plate to be affixed to the bone with the use of surgical screws.

Bone grafting is also known. Grafting is the process in which living or non-living bone is placed between or into the living bone of the body. During the process, the graft becomes incorporated into the renewed bone structure of the living bone as it replaces itself and regrows. Over time, the new bone growth replaces the graft and eliminates the graft material regardless of whether the graft is of living or non-living bone tissue or other material which is resorbable by the body, e.g., a resorbable polymer, a composite, etc.

Heretofore, bone fixation and bone grafting have been separate operations. Bone fixation involved the use of metallic plates affixed to the surface of a fractured or broken bone to hold the separate portions of the bone in place during the healing process. Meanwhile, bone grafting involved the use of pins, nails or screws which enter the bone and hold the bone together thereby allowing regrowth of new bone and healing. Accordingly, a need exists for a bone plate which is fabricated from a material which can be remodeled by the body, e.g., partially demineralized or fully mineralized bone, osteoconductive polymer, composite, etc., which can be affixed to the exterior of a fractured bone and will be grafted to the fracture or break site.

### BRIEF SUMMARY OF THE INVENTION

It is an object of the invention to provide a bone plating system in the form of an external fixation plate for uniting and immobilizing bone with the fixation plate being eventually remodeled and incorporated into the recipient's bone at the site of implantation.

It is another object if this invention to provide a bone plating system made from human cortical bone or xenograft bone sources.

It is yet another object of this invention to provide a bone plating system that is attached with a bone screw that is made from human cortical bone, xenograft cortical bone or other biocompatible material.

It is yet a further object of the invention to provide a bone plating system wherein two or more subunits are assembled into an integrated external fixation plate.

It is still a further object of this invention to provide a bone plating system that is specifically configured for surgical implantation in the subject host, or recipient.

It is another object of the invention to affix the bone plating system to the exterior surface of bone elements, or bone portions, which are to be united and/or immobilized.

It is another object of the invention to provide a preformed recess in the recipient's bone for receiving the bone plating system.

It is yet another object of the invention to provide the bone plating system with preformed apertures for receiving bone screws or other fixation devices.

It is a further object of the invention to provide a bone plating system wherein only portions of the bone plate have been partially demineralized to provide selective flexibility to pre-designated areas of the bone plate.

In keeping with the foregoing objects, a bone plating system is provided which comprises an external fixation plate fabricated from one or more materials capable of remodeling with a recipient's bone such that the fixation plate will initially unite and/or immobilize recipient's bone and ultimately be remodeled and incorporated permanently at the fixation site.

The material utilized in the fabrication of the bone plating system of this invention must be capable of remodeling with the recipient's own bone. Suitable materials include mineralized bone, demineralized bone, osteoconductive polymers and composites, etc., and combinations thereof. The bone plating system is remodeled and replaced by new host bone as incorporation of the bone progresses in vivo. As described more fully below, when bone is utilized as the material for the bone plating system the bone can be fully demineralized by removing substantially all of its inorganic mineral content, the bone can be partially demineralized by removing a significant amount, but less than all, of its inorganic mineral content, or the bone can be only superficially demineralized by removing a minor amount of its inorganic mineral content.

The expression “bone plating system” as utilized herein is to be understood in its broadest sense and is not intended to be limited to any particular shape, configuration or dimension.

Mineralized bone provides strength to the fixation plate and allows the plate to initially support load. Demineralized bone induces new bone formation at the site of the demineralized bone and permits adjustment of the overall mechanical properties of the fixation plate. Therefore, it may be desirable to fabricate a fixation plate from partially demineralized bone, i.e., bone which retains most of its mineral content and therefore most of its mechanical strength, and at the same time a degree of demineralization which renders the bone more biologically active and able to remodel more quickly with the recipient's bone. Where partially demineralized bone is employed, it is generally advantageous to demineralize just the surface of the bone so as to optimize the aforementioned properties of mechanical strength and biological (remodeling) activity.

The term “demineralized” as used herein refers to bone containing less than its original content and is intended to encompass such expressions as “substantially demineralized”, “partially demineralized” and “fully demineralized”.

As utilized herein, the expression “superficially demineralized” refers to bone-derived elements possessing at least about 90 weight percent of their original mineral content, the expression “partially demineralized” refers to bone derived elements possessing from about 8 to about 90 weight percent of their original inorganic mineral content and the expression “fully demineralized” refers to bone containing less than 8% of its original mineral context.

The term “osteogenic” as applied to the bone of this invention shall be understood as referring to the ability of the bone to enhance or accelerate the ingrowth of new bone tissue by one or more mechanisms such as osteogenisis, osteoconduction and/or osteoinduction.

The term “osteoinductive” as used herein shall be understood to refer to the ability of a substance to recruit cells from the host which have the potential for repairing bone tissue.

The term “osteoconductive” as used herein shall be understood to refer to the ability of a substance to provide biologically inert surfaces which are receptive to the growth of new host bone.

The term “osteogenesis” shall be understood to refer to the mechanism by which a non-osteoinductive substance serves as a suitable template or substrate along which bone may grow.

The term “shaped” as applied to the bone plate herein refers to a determined or regular form or configuration, in contrast to an indeterminate or vague form or configuration (as in the case of a lump or other solid mass of no special form) and characteristic of materials such as sheets, plates, disks, cones, pins, screws, tubes, teeth, bones, portion of bone, wedges, cylinders, threaded cylinders, and the like.

### BRIEF DESCRIPTION OF THE DRAWINGS

FIGS. 1-4 illustrate various embodiments of the bone plating system of the invention.

### DETAILED DESCRIPTION OF THE PREFERRED EMBODIMENTS

Preferred embodiments of the disclosed bone plating system will now be described in detail with reference to the drawings, in which reference numerals designate the identical or corresponding elements in each of the several views.

Preferably, each of the fixation plates constituting the bone plating system of the invention is formed from cadaveric human or animal cortical bone. Alternatively, and as previously indicated, the fixation plate can be constructed from one or more other biocompatible materials including ceramics, polymers, composites and the like, which can be remodeled by the body following implantation of the fixation plate. Significant advantages accrue to such materials, and especially bone-derived materials, compared with metallic implants and implants fabricated from other nonresorbable, nonremodelable materials. For example, in the case of bone, the bone plate will eventually remodel with the bone to which it is applied, a capability not possessed by a metal or other nonresorbable material. Additionally, since the bone plate will eventually be replaced by natural bone tissue from the recipient, stress-shielding of the bone at the implantation site, a problem with metal implants, will be avoided. Stress shielding refers to the fact that a metal implant will carry stress rather than the adjacent bone, thus the bone in contact with the metal bone plate will not be as strong in carrying loads or when placed under stress. Further, should the bone plate need to be removed for any reason, a bone or bone-derived bone plate will be easier to remove, with less removal of additional tissue, than the removal of a metal bone plate which would require the removal of additional healthy tissue.

In a preferred embodiment of the invention, the bone plate is placed within a preformed recess in the recipient's bone at the implantation site. This provides a more predictable and secure placement of the bone plate. Moreover, during the process of forming a preformed recess, weak spots in the bone may be identified, and the resulting bone plate will be better suited for bearing weight. In addition, the preformed recess permits more immediate access of the bone plate to cancellous or “soft” bone tissue where the majority of regenerative bone cells are located, thus accelerating fusion of the bone plate with the adjacent bone.

The bone used for making the fixation plate can be partially or fully demineralized. Demineralized bone is generally obtained by taking cortical allograft bone and removing the surface lipids and dehydrating the bone with ethanol and ethyl ether. The bone is then processed with hydrochloric acid. This process removes acid-soluble proteins in the bone, leaving behind protein, bone growth factors, and collagen. Alternatively, other known methods of bone demineralization may be used. The demineralized bone resulting from the process is both osteoconductive and osteoinductive. By increasing the osteoconductive and osteoinductive characteristics of the implant, i.e., bone plate, the fusion of the implant to the bone can be accelerated.

The fixation plate is intended to be applied at a bone repair site, e.g., one resulting from injury, defect brought about during the course of surgery, infection, malignance or developmental malformation. The fixation plate can be utilized in a wide variety of orthopedic, periodontal, neurosurgical and oral and maxillofacial surgical procedures such as the repair of simple and compound fractures and non-unions, external and internal fixations, joint reconstructions such as arthrodesis, general ardiroplasty; cup arthroplasty of the hip, femoral and humeral head replacement; femoral head surface replacement and total joint replacement; repairs of the vertebral column including spinal fusion and internal fixation; tumor surgery, e.g., deficit filling, disectomy; laminectomy; excision of spinal cord tumors, anterior cervical and thoracic operations, repairs of spinal injuries, scoliosis, lodosis, and kyphosis treatments; intermaxillary fixation of fractures; mentoplasty, temporomandibular joint replacement; alveolar ridge augmentation and reconstruction, inlay osteoimplants; implant placement and revision, sinus lifts; cosmetic procedures; etc. Specific bones which can be repaired or replaced utilizing the fixation plate of the invention include the ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, ilium, ischium, pubis, femur, tibia, fibula, patella, calcaneus, tarsal and metatarsal bones.

Clinical applications of the fixation plate include, e.g., the treatment of traumatic fractures, pathologic fractures, stress fractures, congenital defects or fractures or operative defects in any bone of the body that is amenable to fixation. Fracture categories treated with the fixation plate can include, but are not limited, to intraarticular or periarticular fractures; metaphyseal fractures; transverse, oblique comminuted and fragmented fractures; repair to non-fractured sites defects due to periodontal disease or surgery; and other bone defects.

Optional materials can also be incorporated into the fixation plate prior to or after its placement at the surgical site. For example, at the time just prior to when the fixation plate of the invention is to be implanted at a defect site, optional materials, e.g., autograft bone marrow aspirate, autograft bone, preparations of selected autograft cells, autograft cells containing genes or genes encoding bone promoting action or any other agent which induces or accelerates appropriate healing, etc., can be incorporated into the fixation plate.

The fixation plate can be implanted at the bone repair site, if desired, using any suitable affixation means, e.g., sutures, staples, bioadhesives, screws, pins, rivets, other fasteners, and the like, or the plate can be retained in place by the closing of soft tissues around it.

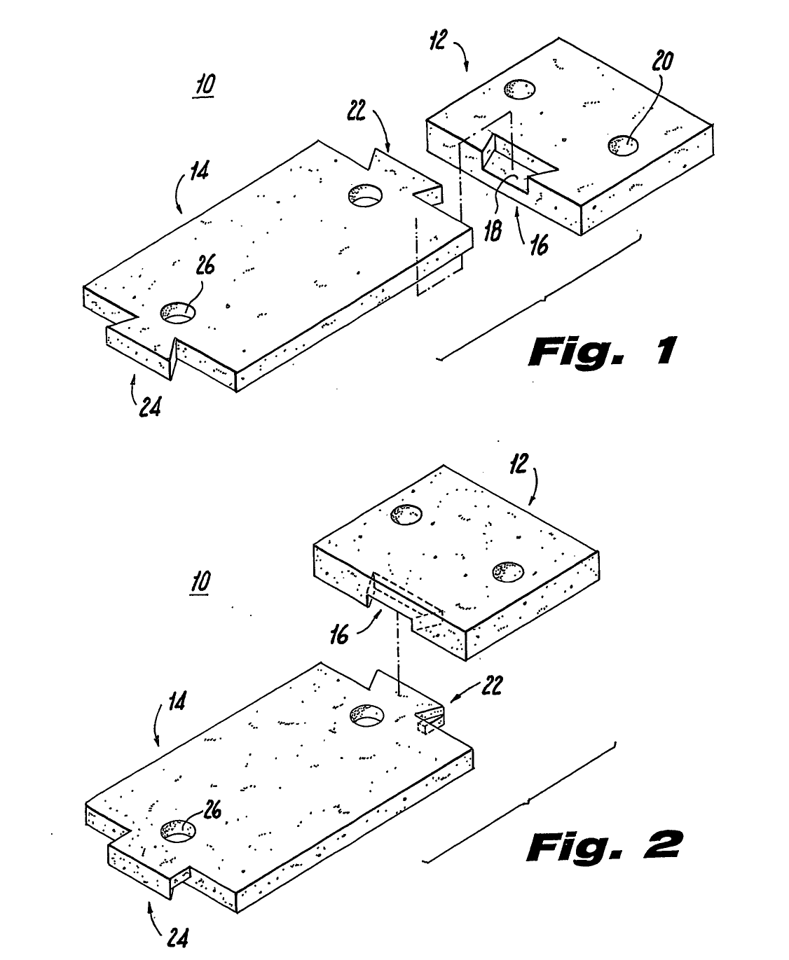

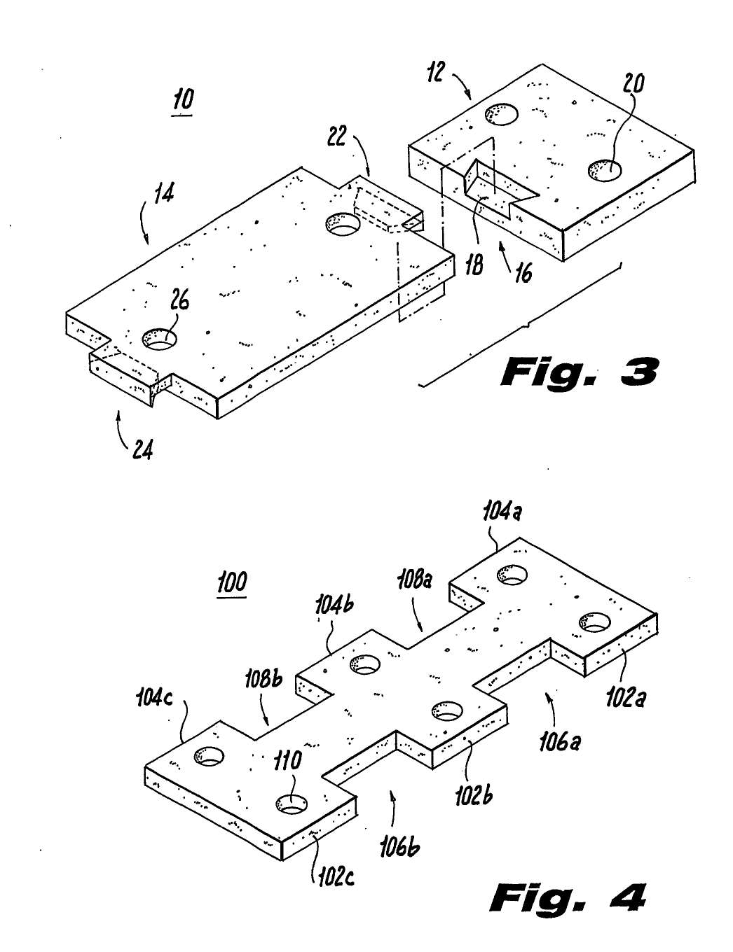

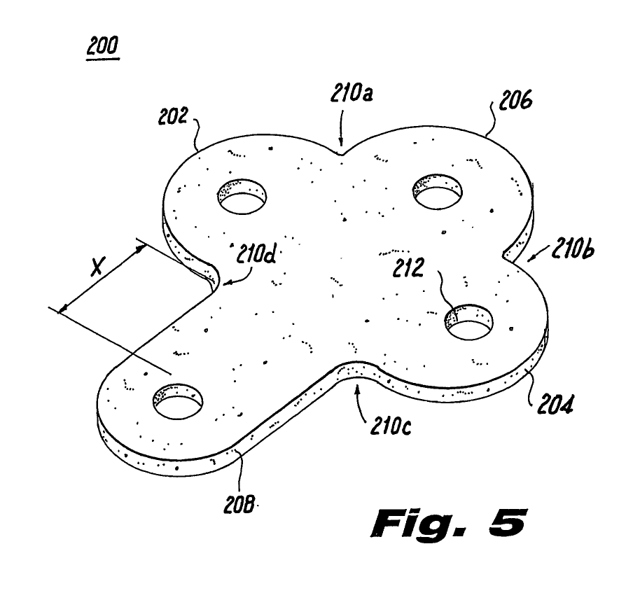

Referring now to the figures, FIGS. 1-3 represent a first preferred embodiment of a cortical bone fixation plate 10 according to the present invention. Fixation plate 10 comprises a first body portion 12 and a second body portion 14. First body portion 12 is generally rectangular in shape and is provided with a trapezoidal recess 16 formed in its surface and along a side thereof. Trapezoidal recess 16 extends partially into the surface of first body portion 12 defining a base wall 18. The first body portion is further provided with at least one throughbore 20 adapted for receiving a threaded screw (not shown) for affixing the first body portion to a bone. Throughbore 20 may be threaded to engage the screw or it may be dimensioned to allow the screw to pass freely therethrough.

Second body portion 14 is generally rectangular in shape and is provided with a trapezoidal projection 22 extending from a side thereof. Trapezoidal projection 22 of second body portion 14 possesses a size and thickness substantially equal to the size and depth of trapezoidal recess 16 in first body portion 12. Second body portion 14 is further provided with a trapezoidal projection 24 extending from a side opposite trapezoidal projection 22 and with at least one throughbore 26. In this manner, another first body portion 12 possesing a corresponding trapezoidal recess 16 can be joined to the side opposite trapezoidal projection 22.

Similar to a dove tail joint, trapezoidal projection 22 of the second body portion 14 is inserted into trapezoidal recess 16 of first body portion 12 thereby joining second body portion 14 to first body portion 12. Although not shown, each of first and second body portions 12 and 14 may include a plurality of projections and/or recesses to facilitate attachment to a plurality of different body portions. In this manner, several bone fixation plates 10 can be attached to one another either side-by-side or end-to-end to create a bone fixation plate having a desired width and/or desired length for securing multiple vertebrae and/or bone elements in relation to each other.

As discussed above, bone fixation plate 10 can be formed from any biocompatible material including those listed above, however, it is preferred that the plate be formed from cortical bone, especially human cortical bone.

While FIGS. 1-3 disclose a first body portion 12 of a bone plate 10 having a trapezoidal recess 16 adapted for receiving a corresponding trapezoidal projection 22 of a second body portion 14, it is contemplated that a variety of other geometries can be utilized for joining the first body portion to the second body portion. In addition, while FIGS. 1-3 show a trapezoidal recess 16 having a depth less than the full thickness of the first body portion 12, trapezoidal recess 16 can have a depth equal to the thickness of first body portion 12 and trapezoidal projections 22 and 24 can have a thickness substantially equal to the depth of trapezoidal recess 16.

FIG. 4 illustrates an alternate embodiment of the presently disclosed cortical bone fixation plate shown generally as 100. Fixation plate 100 is generally rectangular in shape. Plate 100 includes a first set of tabs 102a, 102b and 102c extending from a first side thereof and a second set of tabs 104a, 104b and 104c extending from a second side opposite the first side. Each tab 102a, 102b and 102c of the first set of tabs defines a recess 106a and 106b therebetween. Likewise, each tab 104a, 104b and 104c of the second set of tabs defines a recess 108a and 108b therebetween. Bone plate 100 further includes a number of apertures 110 formed near each of the tabs 102a, 102b, 102c, 104a, 104b and 104c.

For illustrative purposes, in use, the long axis of bone plate 100 is substantially aligned with the long axis of a bone or bones, e.g., the vertebrae or a fractured or broken bone. Tabs 102a, 102b, 102c, 104a, 104b and 104c are then effectively urged against the surface of the bone(s) and the screws (not shown) are inserted into the bone via the apertures 110 thereby securing bone fixation plate 100 to the bone. With plate 100 secured in place, the grafting process of the plate to the bone can begin as a result of the osteoconductive and osteoinductive nature of the plate. Moreover, the adjoining bones or bone elements/bone portions are maintained in fixed relation to allow fusion to occur.

As discussed above, fixation plate 100 can be formed from any remodelable material including any of those listed above; however, it is preferred that the plate be formed from cortical bone.

FIG. 5 illustrates another embodiment of the bone plating system herein, the external fixation plate being shown generally as 200. Bone plate 200 has a generally smooth curved cruciform shape. Bone plate 200 is defined by a left semi-circular arm 202, a right semi-circular arm 204, an upper semi-circular arm 206 and a lower elongated semi-circular arm 208. The lower elongated semi-circular arm 208 extends from the side surface of the left and right semi-circular arms 202 and 204 a selected distance “X.” The distance “X” is dependent on the length of the lower elongated semi-circular arm 208 need to properly secure the two portions of the patients bones. In general, distance “X” can range from about 1.0 to about 5.0 cm and preferably from about 2.0 to about 4.0 cm.

The side surface of left semi-circular arm 202 interconnects with the side surface of upper semi-circular arm 206 via a fillet 210a. The side surface of upper semi-circular arm 206 interconnects with the side surface of right semi-circular arm 204 via a fillet 210b. The side surface of right semi-circular arm 204 interconnects with the side surface of lower elongated semi-circular arm 208 via a fillet 210c. The side surface of lower elongated semi-circular arm 208 interconnects with the side surface of left semi-circular arm 202 via a fillet 210d. While fillets 210a-d are employed to join the side surfaces of the individual arms with one another, other types of connections are possible such as right angle corners, oblique corners, chamfered corners, and the like.

Each of semi-circular arms 202, 204, 206 and 208 are provided with a number of apertures 212 for receiving screws (not shown). Apertures 212 permit fixation plate 200 to be securely affixed to the bone of the recipient.

As discussed above, bone plate 200 can be formed from any biocompatible material including those listed above, however, it is preferred that the bone plate be formed from cortical bone.

It will be understood that various modifications may be made to the embodiments disclosed herein. For example, the particular configuration of the bone plate may be altered to better suit a particular surgical procedure. Moreover, select portions of the bone plate may be partially or fully demineralized to provide flexibility to the bone implant, e.g., projection 22 of body portion 14 of bone plate 10 can be demineralized to accommodate misalignment between body portions 12 and 14 of bone plate 10. Therefore, the above description should not be construed as limiting, but merely as exemplifications of preferred embodiments. Those skilled in the art will envision other modifications within the scope and spirit of the claims appended hereto.

1. A bone plating system which comprises an external fixation plate fabricated from one or more materials capable of remodeling with a mammalian recipient's bone such that the fixation plate will initially unite and/or immobilize recipient's bone and ultimately be remodeled and incorporated permanently at the fixation site.

2. The bone plating system of claim 1 wherein the bone plate is fabricated in whole or in part from autograft, allograft, xenograft, cortical, cancellous or corticocancellous bone.

3. The bone plating system of claim 2, made from human cortical bone.

4. The bone plating system of claim 2, made from xenograft cortical bone.

5. The bone plating system of claim 1, wherein the bone is made from fully mineralized to partially demineralized bone.

6. The bone plating system of claim 1 wherein the fixation plate is substantially planar.

7. The bone plating system of claim 1, wherein the external fixation plate is attached to the site of fixation with a screw made from bone and/or other remodelable material.

8. The bone plating system of claim 7 wherein the other remodelable material is a ceramic, polymer, bone composite or non-bone composite.

9. The bone plating system of claim 1, where several external fixation plates are joined together to provide a desired configuration.

10. The bone plating system of claim 1, where the external fixation plate is configured and fitted to a specific architecture for surgical implantation.

11. The bone plating system of claim 1, wherein the external fixation plate is incorporated into the renewed bone structure of the living bone which is united or immobilized and such bone growth replaces or eliminates the graft material.

12. The bone plating system of claim 1, wherein the external fixation plate is attached to the exterior surface of the bone.

13. The bone plating system of claim 1, wherein the external fixation plate includes a plurality of projections and/or recesses to facilitate attachment to a plurality of different body portions.

14. The bone plating system of claim 1, wherein the external fixation plate accepts a bone screw or other fixation device for attachment to the bone to be united or immobilized.

15. The bone plating system of claim 5, wherein only portions of the bone plate are partially demineralized to impart flexibility to the external fixation plate.

16. The bone plating system of claim 1 adapted to unite or immobilize more then one bone or portions of a single bone.

17. The bone plating system of claim 1 configured to unite or immobilize vertebral segments.

18. A method for the external fixation of bone which comprises affixing to a desired site of bone requiring fixation the bone plating system of claim 1.

19. The method of claim 18 wherein the bone plating system is inserted into a preformed recess within the bone requiring fixation.

20. The method of claim 18 in which the bone plating system is combined with one or more other bone plates and/or osteoimplants to provide a desired implant architecture.
